(19) United States
(12) Patent Application Publication
(10) Pub. No.: US 2025/0295924 A1
(43) Pub. Date: Sep. 25, 2025

(54) SYSTEMS AND METHODS FOR MONITORING AND CONTROLLING AN IMPLANTABLE PULSE GENERATOR

(71) Applicant: Medtronic, Inc., Minneapolis, MN (US)

(72) Inventors: Maneesh Shrivastav, Blaine, MN (US); ShaileshKummar V. Musley, Brooklyn Park, MN (US); Kanthaiah Koka, Valencia, CA (US); Steven M. Goetz, North Oaks, MN (US); Suryakiran Vadrevu, Santa Clarita, CA (US); Rebecca K. Gottlieb, Cluver City, CA (US); David John Miller, Austin, TX (US); Leonid M. Litvak, Los Angeles, CA (US)

(21) Appl. No.: 18/862,801

(22) PCT Filed: Apr. 6, 2023
(86) PCT No.: PCT/US2023/065428

§ 371 Date: Nov. 4, 2024

Related U.S. Application Data
(60) Provisional application No. 63/338,817, filed on May 5, 2022.
(60) Provisional application No. 63/338,794, filed on May 5, 2022.
(60) Provisional application No. 63/338,806, filed on May 5, 2022.
(60) Provisional application No. 63/339,049, filed on May 6, 2022.
(60) Provisional application No. 63/339,101, filed on May 6, 2022.
(60) Provisional application No. 63/339,136, filed on May 6, 2022.
(60) Provisional application No. 63/339,024, filed on May 6, 2022.
(60) Provisional application No. 63/339,304, filed on May 6, 2022.
(60) Provisional application No. 63/339,154, filed on May 6, 2022.
(60) Provisional application No. 63/339,160, filed on May 6, 2022.
(60) Provisional application No. 63/342,945, filed on May 17, 2022.
(60) Provisional application No. 63/342,967, filed on May 17, 2022.
(60) Provisional application No. 63/342,998, filed on May 17, 2022.

(51) Int. Cl. A61N 1/36

(52) U.S. Cl. CPC A61N 1/36132; A61N 1/36053

(57) ABSTRACT

Systems and methods for monitoring an implantable pulse generator are provided. The system may comprise an implantable pulse generator configured to generate a current and an electrode configured to apply the current to an anatomical element. Patient feedback may be monitoring using at least one device and an activation signal may be generated based on the patient feedback. The activation signal may be transmitted to the implantable pulse generator to cause the implantable pulse generator to generate the current, thereby causing the electrode to apply the current to the anatomical element.

### CROSS REFERENCE TO RELATED APPLICATIONS

This application claims the benefit of U.S. Provisional Application No. 63/338,817, filed on May 5, 2022, entitled “Systems and Methods for Monitoring and Controlling an Implantable Pulse Generator”, and further identified as Attorney Docket No. A0008259US01 (10259-211-8P); U.S. Provisional Application No. 63/338,794, filed on May 5, 2022, entitled “Systems and Methods for Stimulating an Anatomical Element Using an Electrode Device”, and further identified as Attorney Docket No. A0008247US01 (10259-211-1P); U.S. Provisional Application No. 63/339,049, filed on May 6, 2022, entitled “Systems and Methods for Mechanically Blocking a Nerve”, and further identified as Attorney Docket No. A0008250US01 (10259-211-2P); U.S. Provisional Application No. 63/338,806, filed on May 5, 2022, entitled “Systems and Methods for Wirelessly Stimulating or Blocking at Least One Nerve”, and further identified as Attorney Docket No. A0008251US01 (10259-211-3P); U.S. Provisional Application No. 63/339,101, filed on May 6, 2022, entitled “Neuromodulation Techniques to Create a Nerve Blockage with a Combination Stimulation/Block Therapy for Glycemic Control”, and further identified as Attorney Docket No. A0008252US01 (10259-211-4P); U.S. Provisional Application No. 63/339,136, filed on May 6, 2022, entitled “Neuromodulation for Treatment of Neonatal Chronic Hyperinsulinism”, and further identified as Attorney Docket No. A0008253US01 (10259-211-5P); U.S. Provisional Application No. 63/342,945, filed on May 17, 2022, entitled “Neuromodulation Techniques for Treatment of Hypoglycemia”, and further identified as Attorney Docket No. A0008255US01 (10259-211-6P); U.S. Provisional Application No. 63/342,998, filed on May 17, 2022, entitled “Closed-Loop Feedback and Treatment”, and further identified as Attorney Docket No. A0008258US01 (10259-211-7P); U.S. Provisional Application No. 63/339,024, filed on May 6, 2022, entitled “Programming and Calibration of Closed-Loop Vagal Nerve Stimulation Device”, and further identified as Attorney Docket No. A0008260US01 (10259-211-9P); U.S. Provisional Application No. 63/339,304, filed on May 6, 2022, entitled “Systems and Methods for Stimulating or Blocking a Nerve Using an Electrode Device with a Sutureless Closure”, and further identified as Attorney Docket No. A0008262US01 (10259-211-11P); U.S. Provisional Application No. 63/339,154, filed on May 6, 2022, entitled “Personalized Machine Learning Algorithm for Stimulation/Block Therapy for Treatment of Type 2 Diabetes”, and further identified as Attorney Docket No. A0008263US01 (10259-211-12P); U.S. Provisional Application No. 63/342,967, filed on May 17, 2022, entitled “Patient User Interface for a Stimulation/Block Therapy for Treatment of Type 2 Diabetes”, and further identified as Attorney Docket No. A0008264US01 (10259-211-13P); and U.S. Provisional Application No. 63/339, 160, filed on May 6, 2022, entitled “Utilization of Growth Curves for Optimization of Type 2 Diabetes Treatment”, and further identified as Attorney Docket No. A0008265US02 (10259-211-14P), all of which applications are incorporated herein by reference in their entireties.

### BACKGROUND

The present disclosure is generally directed to therapeutic neuromodulation and relates more particularly to a stimulation/block therapy to affect glycemic control of a patient.

Diabetes represents a large and growing global health issue with estimates of over 537 million patients worldwide having been diagnosed with type 2 diabetes and estimates of 6.7 million annual deaths related to complications of diabetes. Despite different types of treatments being developed and utilized (e.g., medication, surgery, diet, etc.), type 2 diabetes remains challenging to effectively treat. Type 2 patients must frequently contend with keeping their blood sugar levels in a desirable glycemic range. Prolonged deviations can lead to long term complications such as retinopathy, nephropathy (e.g., kidney damage), cardiovascular disease, etc. Because treatment for diabetes is self-managed by the patient on a day-to-day basis (e.g., the patients self-inject the insulin), compliance or adherence with treatments can be problematic.

### BRIEF SUMMARY

Example aspects of the present disclosure include:

A system for monitoring an implantable pulse generator according to at least one embodiment of the present disclosure comprises an implantable pulse generator configured to generate a current; an electrode configured to apply the current to an anatomical element; a processor; and a memory storing data for processing by the processor, the data, when processed, causes the processor to: monitor patient feedback using at least one device; generate an activation signal when the patient feedback reaches a threshold; and transmit the activation signal to the implantable pulse generator to cause the implantable pulse generator to generate the current, thereby causing the electrode to apply the current to the anatomical element.

Any of the aspects herein, wherein at least one current parameter of the current is adjusted based on the patient feedback.

Any of the aspects herein, wherein the patient feedback comprises at least one of a glucose level, exercise activity, user input, and meal activity.

Any of the aspects herein, wherein the patient feedback comprises a glucose level and the at least one device comprises a continuous glucose monitor configured to track and record the glucose level.

Any of the aspects herein, wherein the threshold comprises a glucose threshold.

Any of the aspects herein, wherein the patent feedback comprises at least one of an exercise and an activity level and the at least one device comprises at least one of an activity sensor and a user device, wherein the user device is configured to receive user input.

Any of the aspects herein, wherein the patient feedback comprises a meal activity, wherein the at least one device comprises at least one of a meal sensor and the user device, and wherein the meal sensor is configured to sense at least one of peristaltic movement, gastric emptying, type of food ingested, and stomach sounds and the user device is configured to receive user input.

Any of the aspects herein, wherein the meal sensor comprises at least one of an electromyography sensing apparatus and a microphone for gastric sound monitoring.

Any of the aspects herein, wherein the activity sensor comprises at least one of a wearable device configured to track an activity, a heart rate monitor, an accelerometer, an altimeter, a blood oxygen level monitor, a bioimpedance sensor, and a skin temperature sensor.

A system for monitoring an implantable pulse generator according to at least one embodiment of the present disclosure comprises an implantable pulse generator configured to generate a current; an electrode configured to apply the current to an anatomical element; a processor; and a memory storing data for processing by the processor, the data, when processed, causes the processor to: receive patient feedback from at least one device; adjust at least one current parameter based on the received patient feedback; and cause the implantable pulse generator to generate the current using the at least one current parameter, thereby causing the electrode to apply the current to the anatomical element.

Any of the aspects herein, wherein the memory stores further data for processing by the processor that, when processed, causes the processor to: determine if the patient feedback meets a threshold, wherein the implantable pulse generator generates the current when the patent feedback meets the threshold.

Any of the aspects herein, wherein the threshold comprises a glucose threshold.

Any of the aspects herein, wherein the patient feedback comprises at least one of a glucose level, exercise activity, a scheduled activation, and meal activity.

Any of the aspects herein, wherein the at least one device comprises a continuous glucose monitor configured to track and record the glucose level.

Any of the aspects herein, wherein the patient feedback comprises an exercise activity, and wherein the at least one device comprises at least one of an activity sensor, a digital calendar, and user input.

Any of the aspects herein, wherein the patient feedback comprises a meal activity, and wherein the at least one device comprises a meal sensor configured to sense at least one of peristaltic movement, gastric emptying, type of food ingested, and stomach sounds.

Any of the aspects herein, wherein the meal sensor comprises at least one of an electromyography and a microphone.

Any of the aspects herein, wherein the activity sensor comprises at least one of a wearable device configured to track an activity, a heart rate monitor, an accelerometer, an altimeter, a blood oxygen level monitor, a bioimpedance sensor, and a skin temperature sensor.

A system for monitoring an implantable pulse generator according to at least one embodiment of the present disclosure comprises an implantable pulse generator configured to generate a current; an electrode configured to apply the current to an anatomical element; a continuous glucose monitor configured to track and record glucose levels; a processor; and a memory storing data for processing by the processor, the data, when processed, causes the processor to: monitor the glucose levels using the continuous glucose monitor; generate an activation signal when the glucose levels reaches a glucose threshold; and transmit the activation signal to the implantable pulse generator to cause the implantable pulse generator to generate the current, thereby causing the electrode to apply the current to the anatomical element.

Any of the aspects herein, wherein the anatomical element comprises one or more vagal trunks.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a diagram of a system according to at least one embodiment of the present disclosure;

FIG. 2 is a block diagram of a system according to at least one embodiment of the present disclosure; and

FIG. 3 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., random-access memory (RAM), read-only memory (ROM), electrically erasable programmable ROM (EEPROM), flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements. The processors listed herein are not intended to be an exhaustive list of all possible processors that can be used for implementation of the described techniques, and any future iterations of such chips, technologies, or processors may be used to implement the techniques and embodiments of the present disclosure as described herein.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

Vagus nerve stimulation (VNS) is a technology that has been developed to treat different disorders or ailments of a patient, such as epilepsy and depression. In some examples, VNS involves placing a device in or on a patient's body that uses electrical impulses to stimulate the vagus nerve. For example, the device may be usually placed under the skin of the patient, where a wire (e.g., lead) and/or electrode connects the device to the vagus nerve. Once the device is activated, the device sends signals through the vagus nerve to the patient's brainstem (e.g., or different target area in the patient, such as other organs of the patient), transmitting information to their brain. For example, with VNS, the device may be configured to send regular, mild pulses of electrical energy to the brain via the vagus nerve. In some examples, the device may be referred to as an implantable pulse generator. An implantable vagus nerve stimulator has been approved to treat epilepsy and depression in qualifying patients.

The vagus nerve (e.g., also called the pneumogastric nerve, vagal nerve, the cranial nerve X, etc.) is responsible for various internal organ functions of a patient, including digestion, heart rate, breathing, cardiovascular activity, and reflex actions (e.g., coughing, sneezing, swallowing, and vomiting). Most patients may have one vagus nerve on each side of their body, with numerous branches running from their brainstem through their neck, chest, and abdomen down to part of their colon. The vagus nerve plays a role in many bodily functions and may form a link between different areas of the patient, such as the brain and the gut. The vagus nerve is a critical nerve for supplying parasympathetic information to the visceral organs of the respiratory, digestive, and urinary systems. Additionally, the vagus nerve is important in the control of heart rate, bronchoconstriction, and digestive processes. In some cases, the vagus nerve may be considered a mixed nerve based on including both afferent (sensory) fibers and efferent (motor) fibers. As such, based on including the two types of fibers, the vagus nerve may be responsible for carrying motor signals to organs for innervating the organs (e.g., via the efferent fibers), as well as carrying sensory information from the organs back to the brain (e.g., via the afferent fibers).

The vagus nerve has a number of different functions. Four key functions of the vagus nerve are carrying sensory signals, carrying special sensory signals, providing motor functions, and assisting in parasympathetic functions. For example, the sensory signals carried by the vagus nerve may include signaling between the brain and the throat, heart, lungs, and abdomen. The special sensory signals carried by the vagus nerve may provide signaling of special senses in the patient, such as the taste sensation behind the tongue. Additionally, the vagus nerve may enable certain motor functions of the patient, such as providing movement functions for muscles in the neck responsible for swallowing and speech. The parasympathetic functions provided by the vagus nerve may include digestive tract, respiration, and heart rate functioning. In some cases, the nervous system can be divided into two areas: sympathetic and parasympathetic. The sympathetic side increases alertness, energy, blood pressure, heart rate, and breathing rate. The parasympathetic side, which the vagus nerve is heavily involved in, decreases alertness, blood pressure, and heart rate, and helps with calmness, relaxation, and digestion.

VNS is considered a type of neuromodulation (e.g., a technology that acts directly upon nerves of a patient, such as the alteration, or “modulation,” of nerve activity by delivering electrical impulses or pharmaceutical agents directly to a target area). For example, as described above, VNS may include using a device (e.g., implanted in a patient or attached to the patient) that is configured to send regular, mild pulses of electrical energy to a target area of the patient (e.g., brainstem, organ, etc.) via the vagus nerve. The electrical pulses or impulses may affect how that target area of the patient functions to potentially treat different disorders or ailments of a patient.

In some examples, for epileptic patients that suffer from seizures, VNS may change how brain cells work by applying electrical stimulation to certain areas involved in seizures. For example, research has shown that VNS may help control seizures by increasing blood flow in key areas, raising levels of some brain substances (e.g., neurotransmitters) important to control seizures, changing electroencephalogram (EEG) patterns during a seizure, etc. As an example, an epileptic patient's heart rate may increase during a seizure or epileptic episode, so the VNS device may be programmed to send stimulation to the vagus nerve regular intervals and when periods of increased heart rate are seen, where applying stimulation at those times of increased heart rate may help stop seizures. Additionally or alternatively, depression has been tied to an imbalance in certain brain chemicals (e.g., neurotransmitters), so VNS is believed to assist in treating patients diagnosed with depression by using electricity (e.g., electrical pulses/impulses) to influence the production of those brain chemicals.

Diabetes represents a large and growing global health issue with estimates of over 537 million patients worldwide having been diagnosed with type 2 diabetes and estimates of 6.7 million annual deaths related to complications of diabetes. Despite different types of treatments being developed and utilized (e.g., medication, surgery, diet, etc.), type 2 diabetes remains challenging to effectively treat. Type 2 patients must frequently contend with keeping their blood sugar levels in a desirable glycemic range. Prolonged deviations can lead to long term complications such as retinopathy, nephropathy (e.g., kidney damage), cardiovascular disease, etc. Because treatment for diabetes is self-managed by the patient on a day-to-day basis (e.g., the patients self-inject the insulin), compliance or adherence with treatments can be problematic. Additionally, in a financial sense, global expenditures for type 2 diabetes treatments, preventive measures, and resulting consequences are estimated at about $966 billion per year. Compounding this issue of high global expenditures is the increasing price of insulin.

As described herein, a neuromodulation technique is provided for glycemic control (e.g., as a treatment for diabetes) using a stimulation/block therapy (e.g., type of VNS). For example, the neuromodulation technique may generally include using a device (e.g., including at least an implantable pulse generator) to provide electrical stimulation (e.g., electrical pulses/impulses) on one or more trunks of the vagus nerve (e.g., vagal trunks) to mute a glycemic response for patients with diabetes. The “patient” as used herein may refer to homo sapiens or any other living being that has a vagus nerve.

In some examples, the device may provide stimulation/blocking of the celiac and hepatic vagal trunks (e.g., using the device) for the purposes of glycemic control. For example, the anterior sub diaphragmatic vagal trunk at the hepatic branching point of the vagus nerve may be electrically blocked (e.g., down-regulated) by delivering a high frequency stimulation (e.g., of about 5 kilohertz (kHz) or in a range between 1 kHz to 50 kHz). Additionally or alternatively, the posterior sub diaphragmatic vagal trunk at the celiac branching point of the vagus nerve may be electrically stimulated (e.g., up-regulated) by delivering a low frequency stimulation (e.g., a square wave at 1Hz or within a range from 0.1 to 20 Hz). In some examples, the electrical blocking and/or electrical stimulating of the respective vagal trunks may be performed by using one or more cuff electrodes (e.g., of the device) placed on the corresponding vagal trunks (e.g., sutured or otherwise held in place). The desired response by providing the stimulation/block therapy is a muting of the glycemic response of a patient. In some examples, muting of the glycemic response may refer to a lower post prandial peak of the glycemic response as compared to a peak without the stimulation/block therapy being applied.

Using the stimulation/block therapy to achieve a muting of the glycemic response is advantageous for those with type 2 diabetes where the postprandial glycemic response (e.g., occurring after a meal) can be very high. For example, some patients with type 2 diabetes may have high blood sugar levels (e.g., glucose levels) after eating a meal based on their reduced or lack of insulin production (e.g., normal insulin production in the body lowers blood sugar levels postprandially by promoting absorption of glucose from the blood into different cells). Additionally or alternatively, patients diagnosed with type 2 diabetes may generally have high glycemic levels at different points of the day (e.g., not necessarily postprandially or immediately after a meal). Over time, the effect of high glycemic values can have a detrimental effect on one's health, leading to neuropathy, retinopathy, and other ailments. Accordingly, by using the stimulation/block therapy provided herein, a high glycemic response experienced by type 2 diabetes patients may be muted (e.g., the glycemic response is reduced, particularly post prandially). Additionally, the therapy aims to improve insulin sensitivity, the lack of which leads to an imbalance in glycemic control and consequent systemic complications in patients with type 2 diabetes. In some examples, the therapy may also improve fasting hyperglycemia, which can be commonly seen in patients with type 2 diabetes.

Thus, embodiments of the present disclosure provide technical solutions to one or more of the problems of (1) controlling a stimulation and/or blocking therapy based on patient feedback, (2) adjusting one or more parameters of a stimulation and/or blocking therapy based on patient feedback (2) automatically controlling a stimulation and/or blocking therapy, (3) increase patient comfort and safety, and (4) incorporating a number of feedback mechanisms to optimize the stimulating and/or blocking therapy.

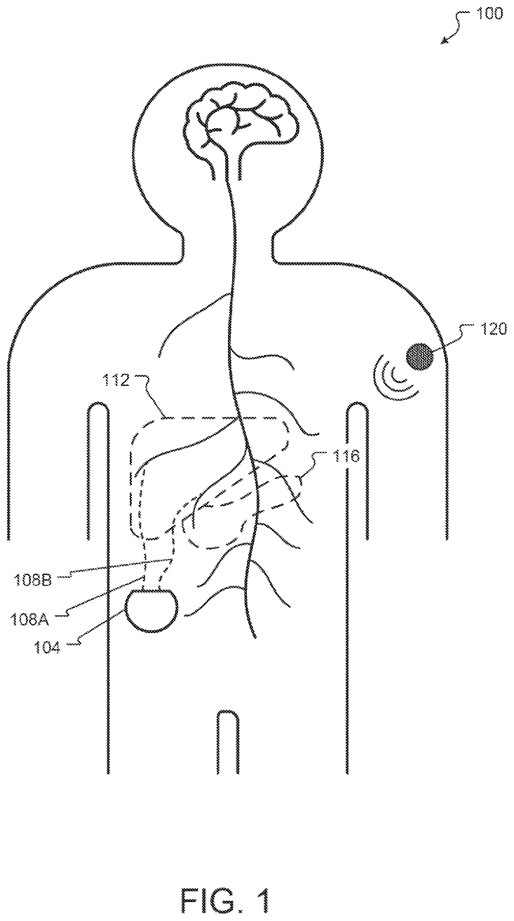

Turning to FIG. 1, a diagram of a system 100 according to at least one embodiment of the present disclosure is shown. The system 100 may be used to provide glycemic control for a patient and/or carry out one or more other aspects of one or more of the methods disclosed herein. For example, the system 100 may include at least a device 104 that is capable of providing a stimulation/blocking therapy that mutes a glycemic response for patients with diabetes. In some examples, the device 104 may be referred to as an implantable pulse generator, an implantable neurostimulator, or another type of device not explicitly listed or described herein. Additionally, the system 100 may include one or more wires 108 (e.g., leads) that provide a connection between the device 104 and nerves of the patient for enabling the stimulation/blocking therapy.

As described previously, neuromodulation techniques (e.g., technologies that act directly upon nerves of a patient, such as the alteration, or “modulation,” of nerve activity by delivering electrical impulses or localized pharmaceutical agents directly to a target area) may be used for assisting in treatments for different diseases, disorders, or ailments of a patient, such as epilepsy and depression. Accordingly, as described herein, the neuromodulation techniques may be used for muting a glycemic response in the patient to assist in the treatment of diabetes for the patient. For example, the device 104 may provide electrical stimulation to one or more trunks of the vagus nerve of the patient (e.g., via the one or more wires 108) to provide the stimulation/blocking therapy for supporting glycemic control in the patient.

In some examples, the one or more wires 108 may include at least a first wire 108A and a second wire 108B connected to respective vagal trunks (e.g., different trunks of the vagus nerve). As described previously, most patients have one vagus nerve on each side of their body, running from their brainstem through their neck, chest, and abdomen down to part of their colon. The vagus nerve plays a role in many bodily functions and may form a link between different areas of the patient, such as the brain and the gut. For example, the vagus nerve is responsible for various internal organ functions of a patient, including digestion, heart rate, breathing, cardiovascular activity, and reflex actions (e.g., coughing, sneezing, swallowing, and vomiting).

Accordingly, the first wire 108A may be connected to a first vagal trunk of the patient (e.g., the anterior sub diaphragmatic vagal trunk at the hepatic branching point of the vagus nerve) to provide an electrical blocking signal (e.g., a down-regulating signal) from the device 104 to that first vagal trunk (e.g., by delivering a high frequency stimulation, such as a given waveform at about 5 kHz). Additionally or alternatively, the second wire 108B may be connected to a second vagal trunk of the patient (e.g., the posterior sub diaphragmatic vagal trunk at the celiac branching point of the vagus nerve) to provide an electrical stimulation signal (e.g., an up-regulating signal) from the device 104 to that second vagal trunk (e.g., by delivering a low frequency stimulation, such as a square wave or other waveform at 1 Hz). By providing the electrical blocking signal and the electrical stimulation signal to the respective vagal trunks, the system 100 may provide a muting of the glycemic response of the patient when the stimulation/blocking therapy is applied. For example, muting of the glycemic response may refer to a lower post prandial peak of the glycemic response as compared to a peak without the stimulation/block therapy being applied.

In some examples, the vagal trunks to which the wires 108 are connected may be connected to or otherwise in the vicinity of one or more organs of the patient, such that the blocking/stimulation signals provided to the respective vagal trunks by the wires 108 and the device 104 are delivered to the one or more organs. For example, the first vagal trunk (e.g., to which the first wire 108A is connected) may be connected to a first organ 112 of the patient, and the second vagal trunk (e.g., to which the second wire 108B is connected) may be connected to a second organ 116. Additionally or alternatively, while the respective vagal trunks are shown as being connected to the corresponding organs of the patient as described, the vagal trunks to which the wires 108 are connected may be connected to the other organ (e.g., the first vagal trunk is connected to the second organ 116 and the second vagal trunk is connected to the first organ 112) or may be connected to different organs of the patient. In some examples, the first organ 112 may represent a liver of the patient, and the second organ 116 may represent a pancreas of the patient. In such examples, the blocking/stimulation signals provided by the wires 108 and the device 104 may be delivered to the liver and/or pancreas of the patient to mute a glycemic response of the patient as described herein.

In some examples, the wires 108 may provide the electrical signals to the respective vagal trunks via electrodes of an electrode device (e.g., cuff electrodes) that are connected to the vagal trunks (e.g., sutured in place, wrapped around the nerves of the vagal trunks, etc.). In some examples, the wires 108 may be referenced as cuff electrodes or may otherwise include the cuff electrodes (e.g., at an end of the wires 108 not connected or plugged into the device 104). Additionally or alternatively, while shown as physical wires that provide the connection between the device 104 and the one or more vagal trunks, the cuff electrodes may provide the electrical blocking and/or stimulation signals to the one or more vagal trunks wirelessly (e.g., with or without the device 104).

Additionally, while not shown, the system 100 may include one or more processors (e.g., one or more DSPs, general purpose microprocessors, graphics processing units, ASICs, FPGAs, or other equivalent integrated or discrete logic circuitry) that are programmed to carry out one or more aspects of the present disclosure. In some examples, the one or more processors may include a memory or may be otherwise configured to perform the aspects of the present disclosure. For example, the one or more processors may provide instructions to the device 104, the cuff electrodes, or other components of the system 100 not explicitly shown or described with reference to FIG. 1 for providing the stimulation/blocking therapy to promote glycemic control in a patient as described herein. In some examples, the one or more processors may be part of the device 104 or part of a control unit for the system 100 (e.g., where the control unit is in communication with the device 104 and/or other components of the system 100).

In some examples, the system 100 may also optionally include a glucose sensor 120 that communicates (e.g., wirelessly) with other components of the system 100 (e.g., the device 104, the one or more processors, etc.) to achieve better glycemic control in the patient. For example, the glucose sensor 120 may continuously monitor glucose levels of the patient, such that if the glucose sensor 120 determines glucose levels are outside a normal or desired range for the patient (e.g., glucose levels are too high or too low in the patient), the glucose sensor 120 may communicate that glucose levels are outside the normal or desired range to the device 104 (e.g., via the one or more processors) to signal for the device 104 to apply the stimulation/blocking therapy described herein to adjust glucose levels in the patient (e.g., mute the glycemic response to lower glucose levels in the patient, block insulin production in the patient as a possible technique to raise glucose levels in the patient, etc.).

The system 100 or similar systems may be used, for example, to carry out one or more aspects of any of the methods described herein. The system 100 or similar systems may also be used for other purposes.

It will be appreciated that the human body has many vagal nerves and the stimulation and/or blocking therapies described herein may be applied to one or more vagal nerves, which may reside at any location of a patient (e.g., lumbar, thoracic, etc.). Further, a sequence of stimulations and/or blocking therapies may be applied to different nerves or portions of nerves. For example, a low frequency stimulation may be applied to a first nerve and a high frequency blockade may be applied to a second nerve.

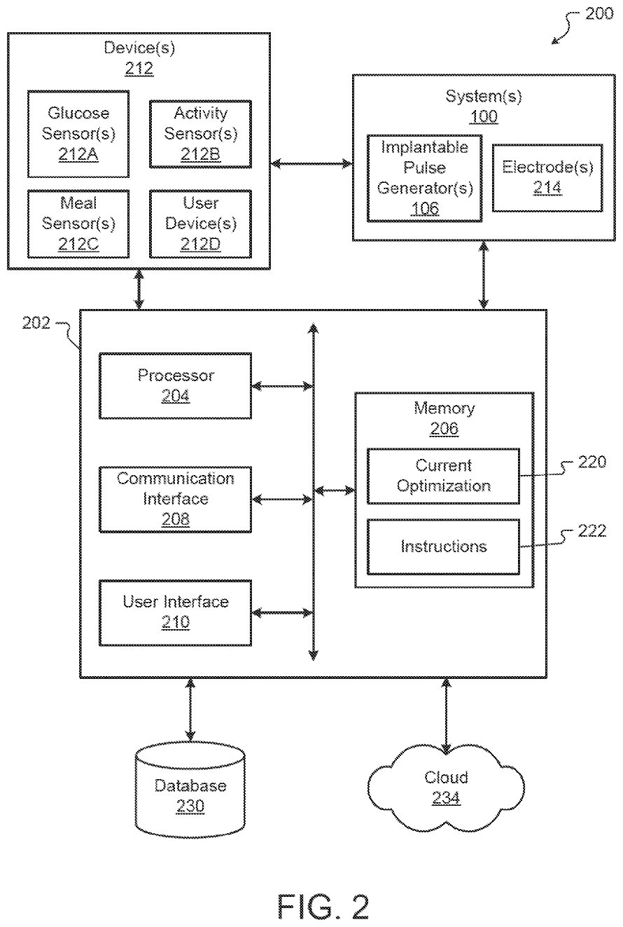

Turning to FIG. 2, a block diagram of a system 200 according to at least one embodiment of the present disclosure is shown. The system 200 may be used with the therapeutic system 100 to provide therapy to a patient, and/or carry out one or more other aspects of one or more of the methods disclosed herein. The system 200 comprises a computing device 202, the therapeutic system 100, one or more device(s) 212, a database 230, and/or a cloud or other network 234. Systems according to other embodiments of the present disclosure may comprise more or fewer components than the system 200. For example, the system 200 may not include one or more components of the computing device 202, the database 230, and/or the cloud 234.

The therapeutic system 100 may comprise the implantable pulse generator(s) 106 and electrode(s) 214. As previously described, the implantable pulse generator 106 may be configured to generate a current. The electrode(s) 214 may be configured to deliver current provided by the implantable pulse generator 106. In particular, the electrode(s) 214 may provide the electrical interface between the wire(s) 104A, 104B and one or more anatomical elements. It should be appreciated the electrode(s) 214 may include one or many individual electrode elements (e.g., contact pads, exposed conductors, etc.). In some embodiments, the system 100 may communicate with the computing device 202 to receive instructions such as instructions 222 for applying a current to the anatomical element. The system 212 may also provide data (such as data received from an electrode 214 capable of recording data), which may be used to optimize the electrode(s) 214 and/or to optimize parameters of the current generated by the implantable pulse generator 106.

The system 200 and/or the therapeutic system 100 may receive patient feedback as input from one or more device(s) 212, which may be used to inform the proper operation of the implantable pulse generator 106 and/or to optimize one or more parameters of the implantable pulse generator 106. For example, the input may be used to determine, optimize, and/or modulate one or more parameters of a current to be generated by the implantable pulse generator 106, when to begin a therapy using the current generated, and a duration of the therapy.

The one or more device(s) may comprise glucose sensor(s) 212A (which may be the same as or similar to the glucose sensor 120), activity sensor(s) 212B, meal sensor(s) 212C, and/or user device(s) 212D. It will be appreciated that the device(s) 212 may comprise more or fewer devices than shown and described. The glucose sensor 212A may be configured to track, record, and/or transmit a patient's glucose level. The patient's glucose level may be checked at regularly-defined intervals (e.g., every 5 minutes). It will be appreciated that the length of these intervals may be increased or decreased, depending upon patient need or care provider preferences. The glucose sensor 212A, in some instances, may be implanted in the patient. In another example, the glucose sensor 212A may be placed subcutaneously.

The activity sensor 212B may be configured to monitor the patient's exercise or activity level. The activity sensor 212B may comprise a wearable device such as, for example, a smart watch configured to track the activity level or exercise of the patient. In some embodiments, the activity sensor 212B may be implanted in the patient. The activity sensor 212B may comprise one or more of a heart rate monitor, an accelerometer, an altimeter, a blood oxygen level monitor, a bioimpedance sensor, a skin temperature sensor, or any combination thereof.

The meal sensor 212C may be configured to track a meal activity. More specifically, the meal sensor 212C may be configured to track when a meal is ingested, what type of meal is ingested, and/or a quantity of the meal ingested. For example, the meal sensor 212C may be configured to sense at least one of a peristaltic movement (to determine when a meal is ingested), gastric emptying, stomach sounds, EMG sensing of the stomach muscles, type of food ingested, and stomach sounds. The meal sensor 212C may also obtain or receive an image of the meal and may use the image to determine information about the meal (e.g., type of meal, quantity of the meal, etc.). In some embodiments, the meal sensor 212C may comprise at least one of an electromyography, a microphone, a hook electrode (which may be, for example, positioned on the stomach to determine muscle stretch), a strain gauge positioned on the esophagus (which may be, for example, used to determine when a meal is ingested) or any combination thereof.

The user device 212D may be configured to receive user input such as, for example, when a meal is ingested, what type of meal, a quantity of the meal, an activity level, exercise, and/or a calendar event (for example, a user may input a 5k race which may indicate that the user will run a 5k race in the future). Further, the user input may comprise a user's personal preferences for the therapeutic treatment and may include input to manually start and/or stop a therapeutic treatment. The user device 212D may comprise, for example, a smart device (e.g., a smart phone, a smart watch, etc.), a computing device (such as, for example, the computing device 202), or the like.

The computing device 202 comprises a processor 204, a memory 202, a communication interface 208, and a user interface 210. Computing devices according to other embodiments of the present disclosure may comprise more or fewer components than the computing device 202.

The processor 204 of the computing device 202 may be any processor described herein or any similar processor. The processor 204 may be configured to execute instructions stored in the memory 202, which instructions may cause the processor 204 to carry out one or more computing steps utilizing or based on data received from the capsule system 212, the database 230, and/or the cloud 234.

The memory 206 may be or comprise RAM, DRAM, SDRAM, other solid-state memory, any memory described herein, or any other tangible, non-transitory memory for storing computer-readable data and/or instructions. The memory 206 may store information or data useful for completing, for example, any step of the method 300 described herein, or of any other methods. The memory 206 may store, for example, instructions and/or machine learning models that support one or more functions of the system 100 and/or the devices 212. For instance, the memory 206 may store content (e.g., instructions and/or machine learning models) that, when executed by the processor 204, enable current optimization algorithm 220.

The current optimization algorithm 220 may correspond to a routine executed by the processor 204 to optimize current used in an electrical stimulation and/or nerve blocking. Optimization may be achieved by adjusting signal frequency, adjusting signal type (e.g., square wave, sinusoidal wave, triangle wave, etc.), adjusting duty cycle, adjusting treatment duration, etc. More specifically, the current optimization algorithm 220 may enable the processor 204 to determine one or more parameters of the current and/or a period of time to generate and apply the current.

Content stored in the memory 206, if provided as in instruction, may, in some embodiments, be organized into one or more applications, modules, packages, layers, or engines. Alternatively or additionally, the memory 206 may store other types of content or data (e.g., machine learning models, artificial neural networks, deep neural networks, etc.) that can be processed by the processor 204 to carry out the various method and features described herein. Thus, although various contents of memory 206 may be described as instructions, it should be appreciated that functionality described herein can be achieved through use of instructions, algorithms, and/or machine learning models. The data, algorithms, and/or instructions may cause the processor 204 to manipulate data stored in the memory 206 and/or received from or via the capsule system 212, the database 230, and/or the cloud 234.

The computing device 206 may also comprise a communication interface 208. The communication interface 208 may be used for receiving data (for example, data from the first capsule 200A and/or the second capsule 200B transmitted via the first communication channel and/or the second communication channel, respectively) or other information from an external source (such as the capsule system 212, the database 230, the cloud 234, and/or any other system or component not part of the system 200), and/or for transmitting instructions, images, or other information to an external system or device (e.g., another computing device 206, the capsule system 212, the database 230, the cloud 234, and/or any other system or component not part of the system 200). The communication interface 208 may comprise one or more wired interfaces (e.g., a USB port, an Ethernet port, a Firewire port) and/or one or more wireless transceivers or interfaces (configured, for example, to transmit and/or receive information via one or more wireless communication protocols such as 206.11a/b/g/n, Bluetooth, NFC, ZigBee, and so forth). In some embodiments, the communication interface 208 may be useful for enabling the device 206 to communicate with one or more other processors 204 or computing devices 206, whether to reduce the time needed to accomplish a computing-intensive task or for any other reason.

The computing device 206 may also comprise one or more user interfaces 210. The user interface 210 may be or comprise a keyboard, mouse, trackball, monitor, television, screen, touchscreen, and/or any other device for receiving information from a user and/or for providing information to a user. The user interface 210 may be used, for example, to receive a user selection or other user input regarding any step of any method described herein. Notwithstanding the foregoing, any required input for any step of any method described herein may be generated automatically by the system 200 (e.g., by the processor 204 or another component of the system 200) or received by the system 200 from a source external to the system 200. In some embodiments, the user interface 210 may be useful to allow a surgeon or other user to modify instructions to be executed by the processor 204 according to one or more embodiments of the present disclosure, and/or to modify or adjust a setting of other information displayed on the user interface 210 or corresponding thereto.

Although the user interface 210 is shown as part of the computing device 206, in some embodiments, the computing device 206 may utilize a user interface 210 that is housed separately from one or more remaining components of the computing device 206. In some embodiments, the user interface 210 may be located proximate one or more other components of the computing device 206, while in other embodiments, the user interface 210 may be located remotely from one or more other components of the computer device 206.

The database 230 may store information such as patient data, results of a stimulation and/or blocking procedure, stimulation and/or blocking parameters, current parameters, electrode parameters, etc. The database 230 may be configured to provide any such information to the computing device 206 or to any other device of the system 200 or external to the system 200, whether directly or via the cloud 234. In some embodiments, the database 230 may be or comprise part of a hospital image storage system, such as a picture archiving and communication system (PACS), a health information system (HIS), and/or another system for collecting, storing, managing, and/or transmitting electronic medical records.

The cloud 234 may be or represent the Internet or any other wide area network. The computing device 206 may be connected to the cloud 234 via the communication interface 208, using a wired connection, a wireless connection, or both. In some embodiments, the computing device 206 may communicate with the database 230 and/or an external device (e.g., a computing device) via the cloud 234.

The system 200 or similar systems may be used, for example, to carry out one or more aspects of any of the method 300 described herein. The system 200 or similar systems may also be used for other purposes.

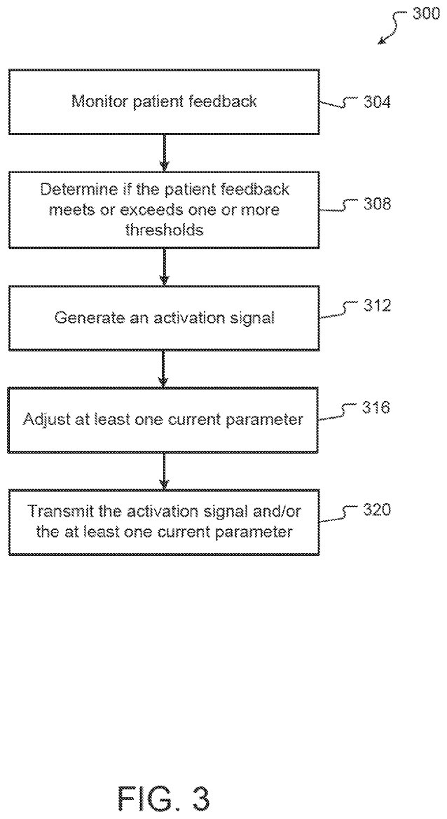

FIG. 3 depicts a method 300 that may be used, for example, for monitoring and/or controlling a therapeutic treatment using an implantable pulse generator. The method 300 beneficially provides for starting and in some instances, adjusting a stimulation and/or blocking therapy based on a user's activities and/or meals ingested. For example, a user may need the stimulation and/or blocking therapy after ingesting certain meals and the method 300 provides for monitoring of such meals and starting and controlling the stimulation and/or blocking therapy as needed.

The method 300 (and/or one or more steps thereof) may be carried out or otherwise performed, for example, by at least one processor. The at least one processor may be the same as or similar to the processor(s) 204 of the computing device 202 described above, or a processor other than any processor described herein may also be used to execute the method 300. The at least one processor may perform the method 300 by executing elements stored in a memory such as the memory 206. The elements stored in the memory and executed by the processor may cause the processor to execute one or more steps of a function as shown in method 300. One or more portions of a method 300 may be performed by the processor executing any of the contents of memory, such as a current optimization algorithm 220 that can be used to optimized one or more parameters of the current.

The method 300 comprises monitoring patient feedback (step 304). The patient feedback may comprise input received from one or more devices. The input may comprise, for example, user input, a meal activity, an exercise activity, an activity level, a glucose level, or any combination thereof. The one or more devices may be the same as or similar to the device(s) 212. The patient feedback may be used to inform the proper operation of an implantable pulse generator such as the implantable pulse generator 106 and/or to optimize one or more parameters of the implantable pulse generator. For example, the input may be used to determine, optimize, and/or modulate one or more parameters of a current to be generated by the implantable pulse generator, when to begin a therapy using the current generated, and a duration of the therapy.

The one or more device(s) may comprise continuous glucose monitor(s) such as the glucose sensor(s) 212A, activity sensor(s) such as the activity sensor(s) 212B, meal sensor(s) such as the meal sensor(s) 212C, and/or user device(s) such as the user device(s) 212D. It will be appreciated that the one or more devices may comprise more or fewer devices than described. As previously described, the continuous glucose monitor may be configured to track, record, and/or transmit a patient's glucose level; the activity sensor may be configured to monitor the patient's exercise or activity level; the meal sensor may be configured to track a meal activity (e.g., when a meal is ingested, what type of meal is ingested, and/or a quantity of the meal ingested); and the user device may be configured to receive user input such as, for example, when a meal is ingested, what type of meal, a quantity of the meal, an activity level, exercise, a calendar event (for example, a user may input a 5k race which may indicate that the user will run a 5k race in the future), a user's personal preferences for the therapeutic treatment, and/or input to manually start and/or stop a therapeutic treatment.

It will be appreciated that the patient feedback may comprise one input (e.g., user input to start a therapeutic treatment) or may comprise a combination of inputs (e.g., an exercise activity input indicating a user exercised and a meal activity indicating a meal ingested by the user after exercising).

The method 300 also comprises determining if the patient feedback meets or exceeds one or more thresholds (step 308). The one or more thresholds may be based on the patient feedback monitored in, for example, the step 304. The one or more thresholds may comprise, for example, a glucose threshold, an activity level threshold, an exercise threshold, and/or a meal threshold. The activity level threshold may comprise, for example, a duration of an activity level, a perceived exertion, a heart rate threshold, or the like. Similarly, the exercise threshold may comprise a heart rate threshold, an exercise duration, a type of exercise, a cadence, or the like. The meal threshold may comprise, for example, a food type, a meal duration, a food quantity, time of day the meal is ingested, or the like.

The one or more thresholds may be determined automatically using artificial intelligence and training data (e.g., historical cases) in some embodiments. In other embodiments, the threshold may be or comprise, or be based on, user input received via a user interface such as the user interface 210 and/or the user device. In further embodiments, the threshold difference may be determined automatically using artificial intelligence, and may thereafter be reviewed and approved (or modified) by a surgeon or other user.

The method 300 also comprises generating an activation signal (step 312). In some embodiments, the activation signal may be generated when the patient feedback meets or exceeds one or more thresholds. For example, the activation signal may be generated when a glucose level monitored by the continuous glucose monitor meets or exceeds a threshold, or is otherwise out of a particular range. The activation signal may also be generated when, for example, a user input is received to start a therapeutic treatment. In some embodiments, the user input may be received from the user interface or the user device. The user input may be, for example, user input indicating that a meal has been ingested. In still other embodiments, the activation signal may be generated based on a scheduled activation. For example, a user may schedule a meal or an activity to occur at a specific time and thus, the activation signal may be generated at such time. In some embodiments, the activation signal may be based on a combination of factors. For example, the activation signal may be generated based on a combination of a glucose level meeting or exceeding a threshold and a user indicated that a meal has been ingested. The combination of factors may provide confirmation that therapeutic treatment is needed. For example, an input indicating that a meal has been ingested (for example, sounds of digestion as recorded by a microphone) may confirm that a scheduled meal has occurred.

The activation signal may be generated automatically by a processor such as the processor 204, a processor of a device such as the device 102, or any other processor. The activation signal may be transmitted to the implantable pulse generator.

The method 300 also comprises adjusting at least one current parameter (step 316). In some embodiments, the step 316 may occur at the same times as the step 312. It will be appreciated that in other embodiments, the step 316 may occur before or after the step 312. In still other embodiments, the method 300 may not include the step 316.

The at least one current parameter of a current generated by the implantable pulse generator may comprise, for example, signal frequency, signal type (e.g., square wave, sinusoidal wave, triangle wave, etc.), duty cycle, treatment duration, or the like. The at least one current parameter may be adjusted based on, for example, the patient feedback received in the step 304. In other words, the patient feedback may be used to modulate or adjust a delivery and/or a duration of the current to a patient (e.g., modulating stimulating or blocking therapy). For example, the current may be adjusted based on when a meal is ingested. In other examples, the current may be adjusted based on a number of other variables such as, for example, stress levels of the user, activity or exercise (e.g., metabolites of such activity or exercise (for example, lactate), type of food ingested, and/or genetic complications. Such variables may be tracked by devices such as the devices 212 and may be, for example, confirmed by user input received from the user device or user interface.

In some embodiments, a processor such as the processor 204 may execute a current optimization algorithm such as the current optimization 220 algorithm. Such optimization may determine one or more parameters of the current and a duration of the current based on one or more inputs such as, for example, the patient feedback, whether one or more thresholds were met or exceeded, and/or user input.

The method 300 also comprises transmitting the activation signal and/or the at least one current parameter (step 320). The activation signal and/or the at least one current parameter may be transmitted to the implantable pulse generator to cause the implantable pulse generator to generate the current using the at least one current parameter for a period of time. As previously described, causing the implantable pulse generator to generate the current causes the electrode to apply the current to the anatomical element. In some embodiments, the step 320 may also comprise transmitting a deactivation signal to the implantable pulse generator at an end of the period of time to cause the implantable pulse generator to deactivate.

It will be appreciated that the method 300 or any of the steps 304, 308, 312, 316, and/or 320 may be repeated. For example, the step 308 may be repeated multiple times throughout a day as a patient ingests various food or participates in various activities. Similarly, the step 316 may be repeated to modulate or adjust the at least one current parameter based on the patient feedback.

The present disclosure encompasses embodiments of the method 300 that comprise more or fewer steps than those described above, and/or one or more steps that are different than the steps described above.

As noted above, the present disclosure encompasses methods with fewer than all of the steps identified in FIG. 3 (and the corresponding description of the method 300), as well as methods that include additional steps beyond those identified in FIG. 3 (and the corresponding description of the method 300). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for monitoring an implantable pulse generator comprising:
an implantable pulse generator configured to generate a current;
an electrode configured to apply the current to an anatomical element;
a processor; and
a memory storing data for processing by the processor, the data, when processed, causes the processor to:
monitor patient feedback using at least one device;
generate an activation signal when the patient feedback reaches a threshold; and
transmit the activation signal to the implantable pulse generator to cause the implantable pulse generator to generate the current, thereby causing the electrode to apply the current to the anatomical element.

2. The system of claim 1, wherein at least one current parameter of the current is adjusted based on the patient feedback.

3. The system of claim 1, wherein the patient feedback comprises at least one of a glucose level, exercise activity, user input, and meal activity.

4. The system of claim 1, wherein the patient feedback comprises a glucose level and the at least one device comprises a continuous glucose monitor configured to track and record the glucose level.

5. The system of claim 1, wherein the threshold comprises a glucose threshold.

6. The system of claim 3, wherein the patent feedback comprises at least one of an exercise and an activity level and the at least one device comprises at least one of an activity sensor and a user device, wherein the user device is configured to receive user input.

7. The system of claim 6, wherein the patient feedback comprises a meal activity, wherein the at least one device comprises at least one of a meal sensor and the user device, and wherein the meal sensor is configured to sense at least one of peristaltic movement, gastric emptying, type of food ingested, and stomach sounds and the user device is configured to receive user input.

8. The system of claim 7, wherein the meal sensor comprises at least one of an electromyography sensing apparatus and a microphone for gastric sound monitoring.

9. The system of claim 6, wherein the activity sensor comprises at least one of a wearable device configured to track an activity, a heart rate monitor, an accelerometer, an altimeter, a blood oxygen level monitor, a bioimpedance sensor, and a skin temperature sensor.

10. A system for monitoring an implantable pulse generator comprising:
an implantable pulse generator configured to generate a current;
an electrode configured to apply the current to an anatomical element;
a processor; and
a memory storing data for processing by the processor, the data, when processed, causes the processor to:
receive patient feedback from at least one device;
adjust at least one current parameter based on the received patient feedback; and
cause the implantable pulse generator to generate the current using the at least one current parameter, thereby causing the electrode to apply the current to the anatomical element.

11. The system of claim 10, wherein the memory stores further data for processing by the processor that, when processed, causes the processor to:
determine if the patient feedback meets a threshold,
wherein the implantable pulse generator generates the current when the patent feedback meets the threshold.

12. The system of claim 11, wherein the threshold comprises a glucose threshold.

13. The system of claim 10, wherein the patient feedback comprises at least one of a glucose level, exercise activity, a scheduled activation, and meal activity.

14. The system of claim 12, wherein the at least one device comprises a continuous glucose monitor configured to track and record the glucose level.

15. The system of claim 13, wherein the patient feedback comprises an exercise activity, and wherein the at least one device comprises at least one of an activity sensor, a digital calendar, and user input.

16. The system of claim 13, wherein the patient feedback comprises a meal activity, and wherein the at least one device comprises a meal sensor configured to sense at least one of peristaltic movement, gastric emptying, type of food ingested, and stomach sounds.

17. The system of claim 16, wherein the meal sensor comprises at least one of an electromyography and a microphone.

18. The system of claim 15, wherein the activity sensor comprises at least one of a wearable device configured to track an activity, a heart rate monitor, an accelerometer, an altimeter, a blood oxygen level monitor, a bioimpedance sensor, and a skin temperature sensor.

19. A system for monitoring an implantable pulse generator comprising:
an implantable pulse generator configured to generate a current;
an electrode configured to apply the current to an anatomical element;
a continuous glucose monitor configured to track and record glucose levels;
a processor; and
a memory storing data for processing by the processor, the data, when processed, causes the processor to:
monitor the glucose levels using the continuous glucose monitor;
generate an activation signal when the glucose levels reaches a glucose threshold; and
transmit the activation signal to the implantable pulse generator to cause the implantable pulse generator to generate the current, thereby causing the electrode to apply the current to the anatomical element.

20. The system of claim 18, wherein the anatomical element comprises one or more vagal trunks.
